(19) United States
(12) Patent Application Publication
(10) Pub. No.: US 2017/0027781 A1
(43) Pub. Date: Feb. 2, 2017

(54) Personal Hygiene Kit

(71) Applicant: Lisa Simmons-Jones, Rancho Palos Verdes, CA (US)

(72) Inventor: Lisa Simmons-Jones, Rancho Palos Verdes, CA (US)

(21) Appl. No.: 14/810,542

(22) Filed: Jul. 28, 2015

(51) Int. Cl. A61F 17/00; B65D 85/00; A41D 13/11; B65D 33/24

(52) U.S. Cl. CPC A61F 17/00; B65D 33/24; B65D 85/70; A41D 13/11

(57) ABSTRACT

A personal hygiene kit for inhibiting cross contamination between a user and a public area includes a pouch that has an open end and a flap. The flap is positionable to selectively cover the open end. A plurality of hygiene items is provided and each of the hygiene items is removably positioned within the pouch. Each of the hygiene items may prevent contamination between an object and a user.

### BACKGROUND OF THE DISCLOSURE

### Field of the Disclosure

The disclosure relates to hygiene devices and more particularly pertains to a new hygiene device for inhibiting cross contamination between a user and a public area.

### SUMMARY OF THE DISCLOSURE

An embodiment of the disclosure meets the needs presented above by generally comprising a pouch that has an open end and a flap. The flap is positionable to selectively cover the open end. A plurality of hygiene items is provided and each of the hygiene items is removably positioned within the pouch. Each of the hygiene items may prevent contamination between an object and a user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a kit view of a personal hygiene kit according to an embodiment of the disclosure.

FIG. 2 is a top perspective view of an embodiment of the disclosure.

FIG. 3 is a perspective view of an embodiment of the disclosure.

### DESCRIPTION OF THE PREFERRED EMBODIMENT

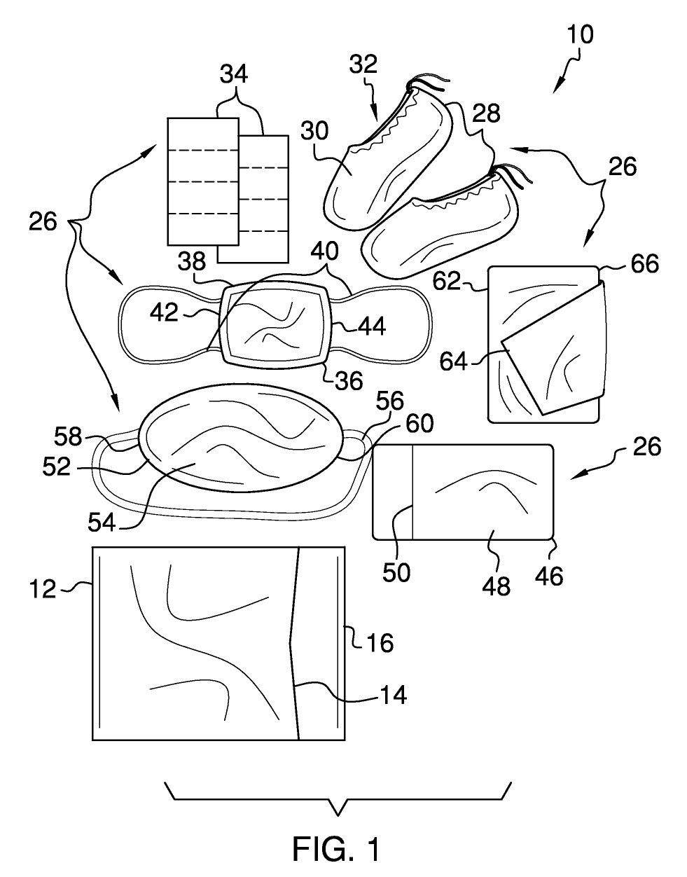

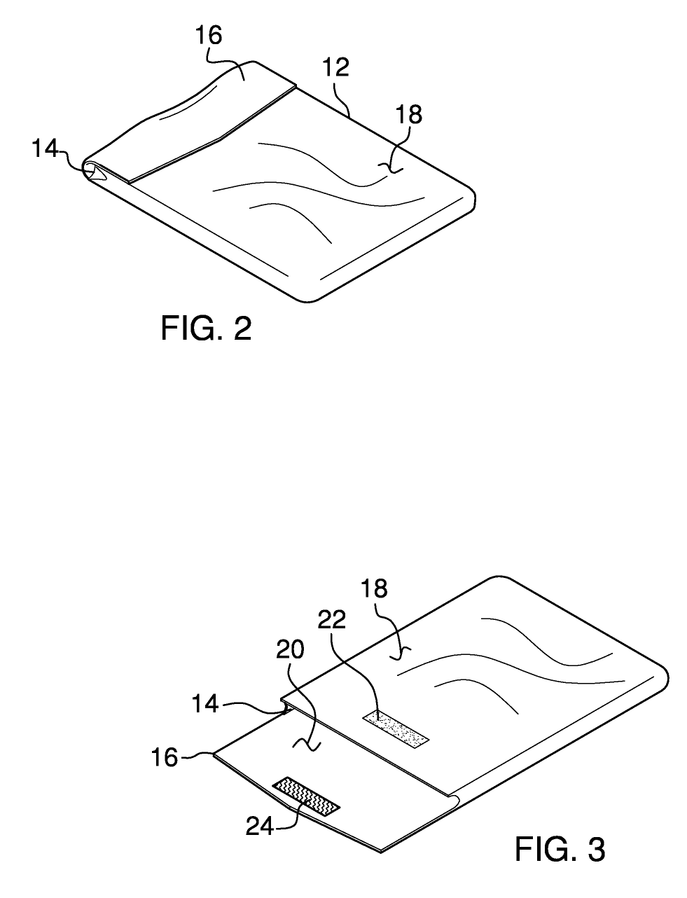

With reference now to the drawings, and in particular to FIGS. 1 through 3 thereof, a new hygiene device embodying the principles and concepts of an embodiment of the disclosure and generally designated by the reference numeral 10 will be described.

As best illustrated in FIGS. 1 through 3, the personal hygiene kit 10 generally comprises a pouch 12 that has an open end 14 and a flap 16. The flap 16 is positionable to selectively cover the open end 14. The pouch 12 has an outer surface 18 and the flap 16 has an engaging surface 20. The engaging surface 20 abuts the outer surface 18 when the flap 16 is positioned to cover the open end 14. The pouch 12 may be comprised of a deformable material such as felt or the like.

A first coupler 22 is coupled to the pouch 12. The first coupler 22 is positioned on the outer surface 18 and the first coupler 22 is positioned adjacent to the open end 14. A second coupler 24 is coupled to the flap 16. The second coupler 24 is positioned on the engaging surface 20. The second coupler 24 is complementary with respect to the first coupler 22 such that the flap 16 is selectively retained in a closed position. Each of the first coupler 22 and the second coupler 24 may comprise a hook and loop fastener or the like.

A plurality of hygiene items 26 is provided and each of the hygiene items 26 is removably positioned within the pouch 12. Each of the hygiene items 26 may prevent contamination between an object and a user. The object may be a public bathroom, public transportation or other area open to public use. The plurality of hygiene items 26 includes a pair of booties 28. Each of the booties 28 has a foot portion 30 and an opening 32 extending into the foot portion 30. Each of the booties 28 may be worn on a foot of a user thereby facilitating each of the booties 28 to prevent cross contamination between a support surface and the foot.

A pair of wipes 34 is provided and each of the wipes 34 is infused with a fluid anti-bacterial. Thus, each of the wipes 34 may selectively disinfect a surface. The fluid anti-bacterial may comprise ethyl alcohol or the like. Each of the wipes 34 may be comprised of a dissolvable material such that each of the wipes 34 dissolves in water.

A mask 36 is provided that has a panel 38 and a pair of loops 40. The panel 38 has a first lateral edge 42 and a second lateral edge 44 and each of the loops 40 is coupled to and extends away from an associated one of the first lateral edge 42 and the second lateral edge 44. Each of the loops 40 may be placed around an ear of user such that the panel 38 substantially covers a face of the user. Thus, the mask 36 inhibits the user from being exposed to respiratory contaminants.

A pillow case 46 is provided and the pillow case 46 has a body 48 and an open end 50. The open end 50 may insertably receive a pillow such that the pillow case 46 completely surrounds the pillow. Thus, the pillow case 46 inhibits the pillow from contaminating the user. A cover 52 is provided that has a pad 54 and a strap 56. The pad 54 has a first side 58 and a second side 60 and the strap 56 is coupled between the first side 58 and the second side 60. The strap 56 may be placed around a headrest such that the pad 54 is retained on the headrest thereby facilitating the pad 54 to inhibit the headrest from contaminating the user. The strap 56 may be comprised of a resiliently stretchable material such as elastic or the like.

A shawl 62 is provided that has a first end 64 and a second end 66. The shawl 62 is elongated between the first end 64 and the second end 66. The shawl 62 may be draped over shoulders of the user thereby facilitating the shawl 62 to inhibit the user's shoulders from becoming contaminated. Additionally, the shawl 62 may be utilized to keep the user warm thereby preventing the user from utilizing blankets intended for public use. Thus, the shawl 62 prevents the blankets intended for public use from contaminating the user.

In use, the pouch 12 is carried when the user expects to utilize a public restroom or public transportation. The booties 28 are worn on the user's feet and the mask 36 is worn on the user's face when the user enters the public restroom. The wipes 34 are utilized to disinfect a surface the user expects to physically contact. The pillow case 46, the shawl 62 and the cover 52 are utilized when the user utilizes public transportation. The mask 36, the booties 28 and the wipes 34 may also be utilized with the user utilizes public transportation.

With respect to the above description then, it is to be realized that the optimum dimensional relationships for the parts of an embodiment enabled by the disclosure, to include variations in size, materials, shape, form, function and manner of operation, kit and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A personal hygiene kit configured for use in a public area, said kit comprising:
a pouch having an open end and a flap, said flap being positionable to selectively cover said open end; and
a plurality of hygiene items, each of said hygiene items being removably positioned within said pouch, each of said hygiene items being configured to prevent contamination between an object and a user.

2. The kit according to claim 1, further comprising said pouch having an outer surface, said flap having an engaging surface, said engaging surface abutting said outer surface when said flap is positioned to cover said open end.

3. The kit according to claim 2, further comprising:
a first coupler being coupled to said pouch, said first coupler being positioned on said outer surface, said first coupler being positioned adjacent to said open end; and
a second coupler being coupled to said flap, said second coupler being positioned on said engaging surface, said second coupler being complementary with respect to said first coupler such that said flap is selectively retained in a closed position.

4. The kit according to claim 1, wherein said plurality of hygiene items includes a pair of booties, each of said booties having a foot portion and an opening extending into said foot portion wherein each of said booties is configured to be worn on a foot thereby facilitating each of said booties to prevent cross contamination between a support surface and the foot.

5. The kit according to claim 1, wherein said plurality of hygiene items further includes a pair of wipes, each of said wipes being infused with a fluid anti-bacterial wherein each of said wipes is configured to selectively disinfect a surface.

6. The kit according to claim 1, wherein said plurality of hygiene items further includes a mask having a panel and a pair of loops, said panel having a first lateral edge and a second lateral edge, each of said loops being coupled to and extending away from an associated one of said first lateral edge and said second lateral edge, each of said loops being configured to be placed around an ear of user such that said panel substantially covers a face of the user thereby facilitating said panel to inhibit to user from being exposed to respiratory contaminants.

7. The kit according to claim 1, wherein said plurality of hygiene items further includes a pillow case having a body and an open end, said open end being configured to insertably receive a pillow such that said pillow case completely surrounds said pillow thereby facilitating said pillow case to inhibit the pillow from contaminating the user.

8. The kit according to claim 1, wherein said plurality of hygiene items further includes a cover having a pad and a strap, said pad having a first side and a second side, said strap being coupled between said first side and said second side, said strap being configured to be placed around a headrest such that said pad is retained on the headrest thereby facilitating said pad to inhibit the headrest from contaminating the user.

9. The kit according to claim 1, wherein said plurality of hygiene items further includes a shawl having a first end and a second end, said shawl being elongated between said first end and said second end, said shawl being configured to be draped over shoulders of the user thereby facilitating said shawl to inhibit the user's shoulders from becoming contaminated.

10. A personal hygiene kit configured for use in a public area, said kit comprising:
a pouch having an open end and a flap, said flap being positionable to selectively cover said open end, said pouch having an outer surface, said flap having an engaging surface, said engaging surface abutting said outer surface when said flap is positioned to cover said open end;
a first coupler being coupled to said pouch, said first coupler being positioned on said outer surface, said first coupler being positioned adjacent to said open end;
a second coupler being coupled to said flap, said second coupler being positioned on said engaging surface, said second coupler being complementary with respect to said first coupler such that said flap is selectively retained in a closed position;
a plurality of hygiene items, each of said hygiene items being removably positioned within said pouch, each of said hygiene items being configured to prevent contamination between an object and a user, said plurality of hygiene items including:
a pair of booties, each of said booties having a foot portion and an opening extending into said foot portion wherein each of said booties is configured to be worn on a foot thereby facilitating each of said booties to prevent cross contamination between a support surface and the foot,
a pair of wipes, each of said wipes being infused with a fluid anti-bacterial wherein each of said wipes is configured to selectively disinfect a surface,
a mask having a panel and a pair of loops, said panel having a first lateral edge and a second lateral edge, each of said loops being coupled to and extending away from an associated one of said first lateral edge and said second lateral edge, each of said loops being configured to be placed around an ear of user such that said panel substantially covers a face of the user thereby facilitating said panel to inhibit to user from being exposed to respiratory contaminants,
a pillow case having a body and an open end, said open end being configured to insertably receive a pillow such that said pillow case completely surrounds said pillow thereby facilitating said pillow case to inhibit the pillow from contaminating the user,
a cover having a pad and a strap, said pad having a first side and a second side, said strap being coupled between said first side and said second side, said strap being configured to be placed around a headrest such that said pad is retained on the headrest thereby facilitating said pad to inhibit the headrest from contaminating the user, and
a shawl having a first end and a second end, said shawl being elongated between said first end and said second end, said shawl being configured to be draped over shoulders of the user thereby facilitating said shawl to inhibit the user's shoulders from becoming contaminated.
